(19) United States
(12) Patent Application Publication
(10) Pub. No.: US 2017/0007830 A1
(43) Pub. Date: Jan. 12, 2017

(54) SMART PORTABLE ELECTROTHERAPY MACHINE

(71) Applicant: U-GYM Technology Corporation, New Taipei City (TW)

(72) Inventors: Fu-Hao YU, New Taipei City (TW); Chin-Chuan HSU, New Taipei City (TW)

(21) Appl. No.: 15/203,017

(22) Filed: Jul. 6, 2016

(30) Foreign Application Priority Data
Jul. 8, 2015 (TW) 104122134

(51) Int. Cl. A61N 1/36; A61N 1/04

(52) U.S. Cl. CPC A61N 1/36014; A61N 1/0492

(57) ABSTRACT

The present invention is constituted by a body, an electric accumulator, an electric connecting wire, an electric quantity display module and an electric current control module. The electric accumulator is connected with the connector, an end of the electric connecting wire is provided with a butting connector which is connected electrically with the connector, and the other end of the electric connecting wire is provided with an electric current transmission element which is installed with a conductive patch. The electric quantity display module is connected electrically with the electric accumulator and the electric current control module is connected with the electric accumulator, so as to control the magnitude and intensity of electric current output in the electric accumulator, and update the electrotherapy mode wirelessly.

### BACKGROUND OF THE INVENTION

a) Field of the Invention

The present invention relates to an electrotherapy machine, and more particularly to a smart portable electrotherapy machine which can be used with terminal management equipment.

b) Description of the Prior Art

An ordinary electrotherapy machine is applied to curing soreness and rehabilitating disorders, wherein an electric wave in a proper waveform, amplitude and frequency is transmitted by electrode plates to stimulate the parts on a patient to be cured.

The existing electrotherapy machines are too huge to be carried, and in order to control an electrotherapy machine, a variety of press buttons is designed according to the intensity of electric current. Therefore, the press buttons will be increased correspondingly when there are too many requirements, and a user needs to understand the function of each press button before controlling the electrotherapy machine. Furthermore, the effect of monitoring can be only achieved through medical personnel or by recording in person. However, this method can easily decrease the accuracy in recording by making a mistake in recording or being unable to record continuously and periodically, due to a human factor. Accordingly, the existing electrotherapy machines are only capable of curing soreness and rehabilitating disorders using an electric current in a low or intermedium frequency.

In addition to the abovementioned issues, an ordinary electrotherapy machine is still in need of improvement regarding the following areas:
1. The electrotherapy machine maintains the conventional design in using the press buttons along with an LCD (Liquid Crystal Display) screen, and therefore all brands of the electrotherapy machine are almost the same, unable to combine with practical application to cause a trend of popularity.
2. Most of the electrotherapy machines are only capable of providing an electric wave in a frequency of a single band and cannot be updated, upgraded or operate at a cross-frequency condition to increase other functions or a practical curative effect. If a consumer needs other functions, he or she will have to purchase a new electrotherapy machine and cannot upgrade the software on the old machine.
3. The electrotherapy is performed in a single-frequency wave mode, which is unable to reach to a deeper part of body and can only stimulate the skin part without being able to stimulate the muscles to contract and move effectively.
4. If the electrotherapy machine is used incorrectly or in an inadequate band of frequency, then the patient may easily feel uncomfortable by getting an electric shock.
5. The ordinary portable electrotherapy machine is too large to be carried for use.

The detailed features and advantages of the present invention are described in details hereinafter, with the content being enough to make anyone familiar with the relevant skills to understand the technical content of the present invention and to implement accordingly. In accordance with the content, the claims and the drawings disclosed in this specification, any person skilled in the relevant art can easily understand the related objects and advantages of the present invention.

### SUMMARY OF THE INVENTION

The primary object of the present invention is to provide a smart portable electrotherapy machine, wherein an electric connecting wire with a conductive patch is used to carry out a physical therapy in low or intermedium frequency, which can massage deeper muscles and soothe physiques. In addition, an ordinary charging cable for transmitting an electric current can be also used to recharge an electronic product. To achieve the abovementioned object, the present invention discloses a smart portable electrotherapy machine including a body which defines a holding space, at least an installing opening connecting the holding space, a display area and a control area; an electric accumulator which is installed in the holding space and is connected electrically with at least a connector mounted at the installing opening; at least an electric connecting wire, an end of which is provided with a butting connector to connect electrically the connector, and the other end of which is provided with at least an electric current transmission element; an electric quantity display module which is installed at the body, disposed in the display area and connected electrically with the electric accumulator; and an electric current control module which is installed at the body, disposed in the control area and connected electrically with the electric accumulator.

According to an embodiment of the present invention, the electric current transmission element is provided with a conductive patch.

According to an embodiment of the present invention, the electric current control module includes at least a control element and a circuit board which is connected with the control element, with the circuit board connecting the electric accumulator.

According to an embodiment of the present invention, the smart portable electrotherapy machine further includes a processor which is installed on the circuit board, a power amplifier which is connected with the processor, and a transformer which is connected with the power amplifier.

According to an embodiment of the present invention, the smart portable electrotherapy machine further includes a g-sensor which is installed on the circuit board and is connected electrically with the processor.

According to an embodiment of the present invention, the connector is a USB (Universal Serial Bus) 2.0 socket, a USB 3.0 socket, a MICRO USB socket or an AUDIO JACK socket.

According to an embodiment of the present invention, the butting connector is a USB 2.0 plug, a USB 3.0 plug, a MICRO USB plug or an AUDIO JACK plug.

In addition to the abovementioned, the present invention discloses a smart portable electrotherapy machine, including a body which defines a holding space and at least an installing opening connecting the holding space, with a surface of the body defining a display area and a control area; an electric accumulator which is installed in the holding space and is connected electrically with at least a connector mounted at the installing opening; at least an electric connecting wire, an end of which is provided with a butting connector to connect electrically the connector, and the other end of which is provided with at least an electric current transmission element; an electric quantity display module which is installed at the body, disposed in the display area and connected electrically with the electric accumulator; an electric current control module which is installed at the body, disposed in the control area and connected electrically with the electric accumulator, with the electric current control module including at least a control element, a circuit board connecting the control element, a processor installed on the circuit board, a power amplifier connecting the processor and a transformer connecting the power amplifier, and the circuit board being provided with a first wireless transmitter connecting electrically the processor; and in particular, also a g-sensor which is installed on the circuit board and connected electrically with the processor; and a terminal management equipment which is built with a second wireless transmitter butting with the first wireless transmitter and is loaded with an application.

According to another embodiment of the present invention, the application is a mobile application (APP).

According to another embodiment of the present invention, the processor is loaded with an AirSig application which is butted with the g-sensor.

A secondary object of the present invention is to disclose a smart portable electrotherapy machine, an entire size of which is only 12.4×5.8 cm to be carried easily. In addition, there are no conventional display screen and complex press buttons, and the function can be changed easily and quickly only through the electric quantity display module and the electric current control module to select an adequate frequency for the deep electrotherapy mode, along with that the power consumption can be known intuitively.

Another object of the present invention is to disclose a smart portable electrotherapy machine which is provided with an ordinary USB 2.0 socket, USB 3.0 socket, MICRO USB socket or other connector fitting with a mobile device as an ordinary mobile power bank, achieving the benefit of convenience and multi-tasking.

Still another object of the present invention is to disclose a smart portable electrotherapy machine, an interior of which is built with a g-sensor to record the user's status of motion at any time, such as distance, speed, height and time of motion. Furthermore, the g-sensor is combined with the usage information of electrotherapy, such as mode, location and time, so that the best combination of usage can be known to achieve a pre-determined goal of health management.

Yet still another object of the present invention is to disclose a smart portable electrotherapy machine, wherein an APP which is built into a terminal management equipment can transmit information wirelessly and directly with the electrotherapy machine through a Bluetooth device and update the electrotherapy mode through the Bluetooth device. In addition, the APP can also record the related information after electrotherapy, and achieve the goal of managing health by the APP.

Again, still another object of the present invention is to disclose a smart portable electrotherapy machine, wherein an AirSig application which is built into a terminal management equipment can be used as a mechanism to enhance the security of personal data, preventing personal data or related personal health records from being stolen. To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a three-dimensional schematic view of the present invention.

FIG. 2 shows a block diagram of the present invention.

FIG. 3 shows a block diagram of the present invention which is connected with terminal management equipment.

FIG. 4 shows a flow diagram of the present invention which is loaded with an APP.

FIG. 5 shows a flow diagram of the present invention which is loaded with an AirSig application.

### DETAILED DESCRIPTION OF THE PREFERRED EMBODIMENTS

The implementation of the present invention is described hereinafter with specific embodiments, and any person skilled in the relevant art can easily understand the related advantages and benefits of the present invention.

The structure, proportion, size, etc. depicted in the drawings of this specification are only disclosed to match content in the specification for those skilled in the art to understand and read, not intended to limit the implementation of the present invention. Therefore, they have no technical meaning, and any modification in structure, change in proportion or adjustment in size should still fall within the technical content disclosed in the present invention, without affecting the efficacy of the present invention to be generated and the purpose of the present invention to be achieved.

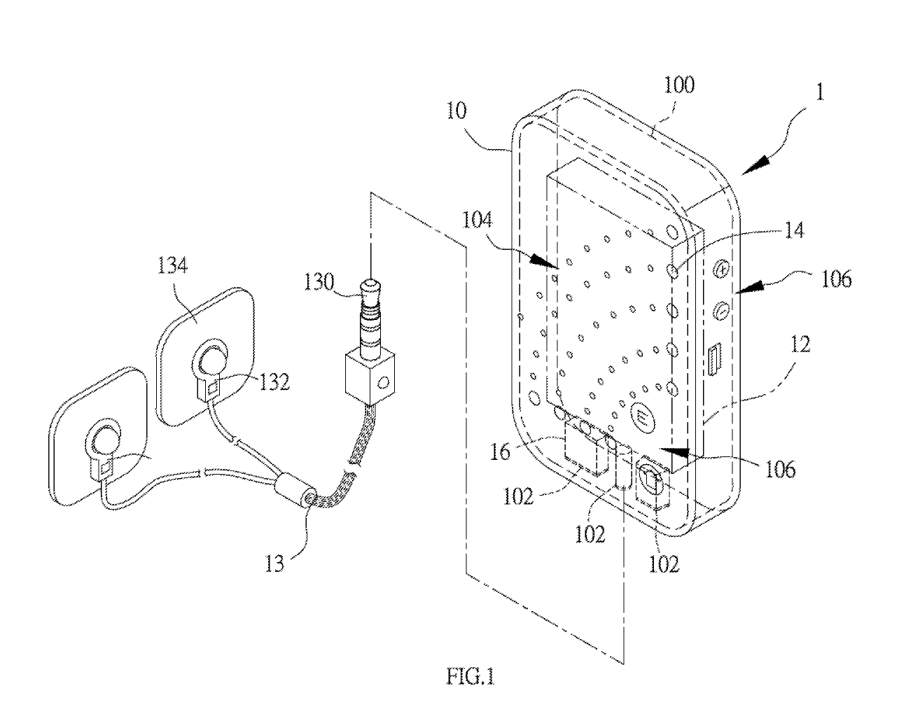

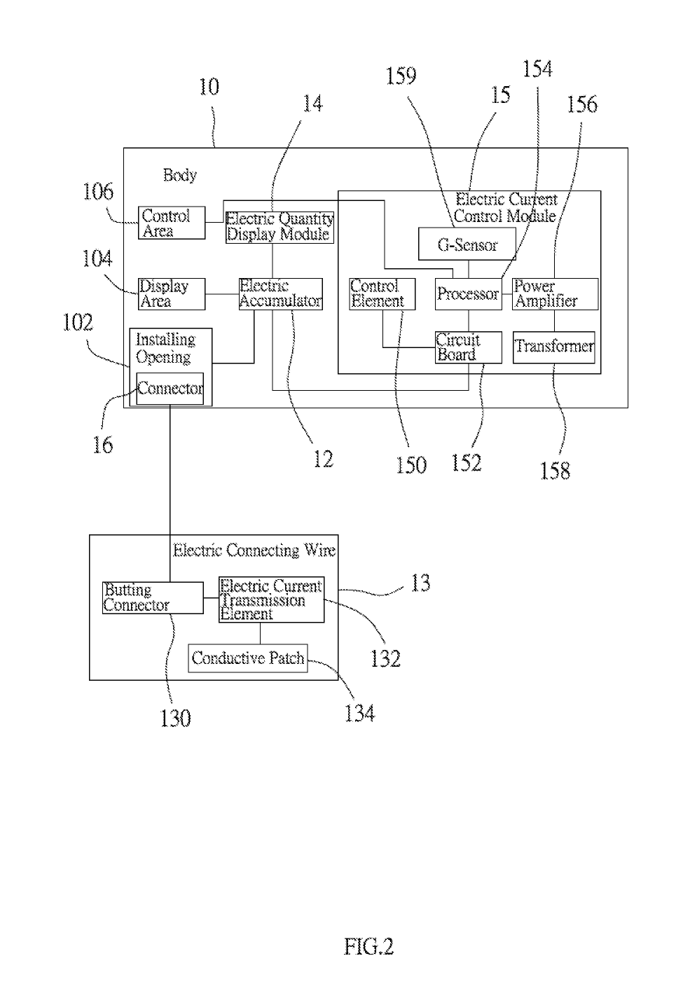

FIG. 1 and FIG. 2 show a three-dimensional schematic view and a block diagram of an electrotherapy machine of the present invention. The present invention discloses an electrotherapy machine 1 which comprises primarily a body 10, an electric accumulator 12, an electric connecting wire 13, an electric quantity display module 14 and an electric current control module 15. The body 10 defines a holding space 100 and at least an installing opening 102 which is connected with the holding space 100. The body 10 also defines a display area 104 and a control area 106; whereas, an interior of the holding space 100 is installed with the electric accumulator 12 that is connected electrically with at least a connector 16 (can be a USB 2.0 socket, a USB 3.0 socket, a MICRO USB socket or an AUDIO JACK socket) mounted at the installing opening 102.

In addition, an interior of the body 10 is installed with the electric quantity display module 14 and the electric current control module 15, the electric quantity display module 14 is disposed in the display area 104 and is connected electrically with the electric accumulator 12, and the electric current control module 15 is disposed in the control area 106 and is connected with the electric accumulator 12. The electric quantity display module 14, on the other hand, can manifest various display methods according to the magnitude of the electric quantity, allowing a user to select an adequate electrotherapy mode. In the present invention, the electric quantity display module 14 uses a fan-shaped white light to show the intensity of electrotherapy energy, an orange light to show the status of electrotherapy, and a blue light to identify the magnitude of electric quantity. Moreover, an end of the electric connecting wire 13 is provided with a butting connector 130 (can be a USB 2.0 plug, a USB 3.0 plug, a MICRO USB plug or an AUDIO JACK plug) to connect electrically the connector 16, and the other end of the electric connecting wire 13 is provided with at least an electric current transmission element 132, with the electric current transmission element 132 being provided with a conductive patch 134.

The abovementioned electric current control module 15 includes at least a control element 150 and a circuit board 152 which is connected with the control element 150. The circuit board 152 is connected with the electric accumulator 12 and is installed with a processor 154, a power amplifier 156 connecting the processor 154, a transformer 158 connecting the power amplifier 156, and a g-sensor 159 installed on the circuit board 152 and connecting electrically the processor 154.

When a user is undergoing the electrotherapy, the butting connector 130 at one end of the electric connecting wire 13 (can be taken as an electrotherapy cable) is inserted into the corresponding connector 16 in the electrotherapy machine 1; in this configuration, the connector 16 and the butting connector 130 can be an AUDIO JACK socket and an AUDIO JACK plug. After insertion, the conductive patch 134 on the electric current transmission element 132 is then attached on the part to be cured, followed by using the control element 150 in the electrotherapy machine 1 to operate and determine the intensity of electric current and the electrotherapy mode. On the other hand, the user can understand the status of the electric accumulator 12 in the electrotherapy machine 1 through the electric quantity display module 14. In the present invention, the electric quantity display module 14 uses a fan-shaped white light to show the intensity of electrotherapy energy, an orange light to show the status of electrotherapy, and a blue light to identify the magnitude of electric quantity. As the entire size of the electrotherapy machine 1 is only 12.4×5.8 cm, it can be carried easily. In addition, there are no conventional display screen and complex press buttons, and the function can be changed easily and quickly only through the electric quantity display module and the electric current control module, along with that the power consumption can be known intuitively

Furthermore, the electrotherapy machine 1 is installed additionally with another kind of connector 16 (can be a USB 2.0 socket, a USB 3.0 socket or a MICRO USB socket). This connector 16 allows the user to use another kind of electric connecting wire 13 (can be a charging cable) to insert the butting connector 130 (can be a USB 2.0 plug, a USB 3.0 plug or a MICRO USB plug) at one end into the corresponding connector 16 in the electrotherapy machine 1; therefore, the inserted electric connecting wire 13 can be connected with a smart phone, a tablet computer or other mobile device which needs to be recharged to carry out recharging, using the electric current transmission element 132 at the other end.

As the electrotherapy machine is provided with an ordinary USB 2.0 socket, USB 3.0 socket, MICRO USB socket or other connector fitting with the mobile device as an ordinary mobile power bank, the purpose of convenience and multi-tasking can be achieved.

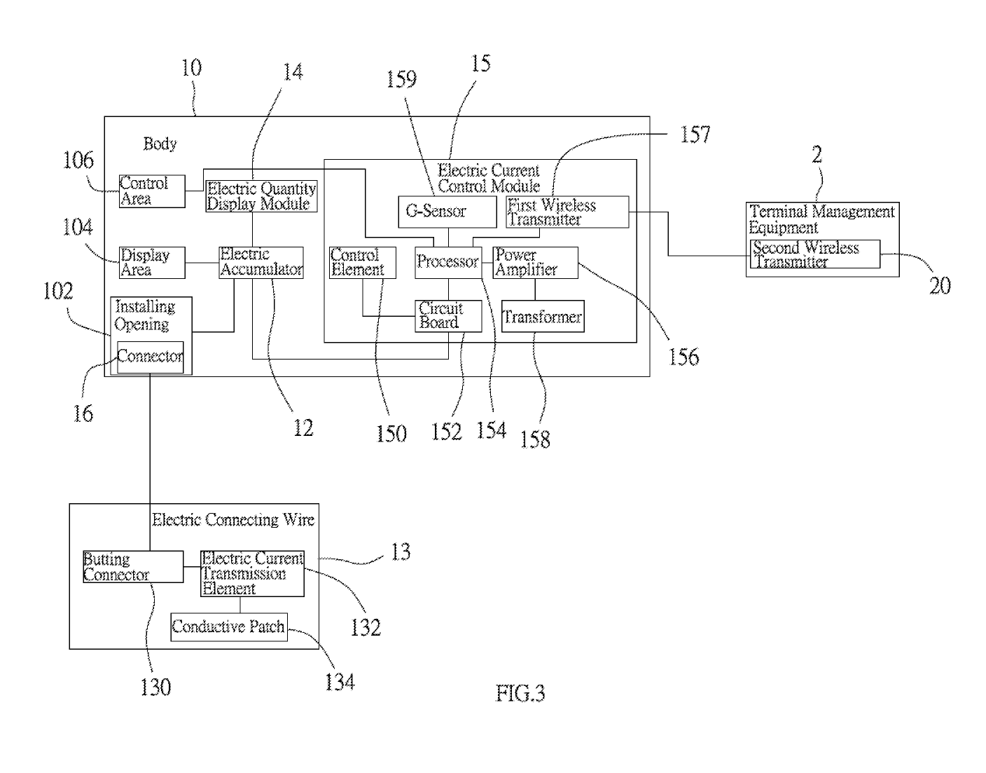

Referring to FIGS. 3 to 5 together, it shows a block diagram of the electrotherapy machine which is connected with terminal management equipment, a flow diagram of the electrotherapy machine which is loaded with an APP, and a flow diagram of the electrotherapy machine which is loaded with an AirSig application. The electrotherapy machine of the present invention can fit with terminal management equipment 2 which can be a tablet computer or a smart phone with network communication.

Referring to FIG. 1 and FIG. 3, the electrotherapy machine 1 is constituted primarily by a body 10, an electric accumulator 12, an electric connecting wire 13, an electric quantity display module 14 and an electric current control module 15. The body 10 defines a holding space 100 and at least an installing opening 102 which is connected with the holding space 100. The body 10 also defines a display area 104 and a control area 106; whereas, an interior of the holding space 100 is installed with the electric accumulator 12 which is connected electrically with at least a connector 16 (can be a USB 2.0 socket, a USB 3.0 socket, a MICRO USB socket or an AUDIO JACK socket) mounted at the installing opening 102.

Moreover, an interior of the body 10 is installed with the electric quantity display module 14 and the electric current control module 15, the electric quantity display module 14 is disposed in the display area 104 and is connected electrically with the electric accumulator 12, and the electric current control module 15 is disposed in the control area 106 and is connected electrically with the electric accumulator 12. The electric quantity display module 14, on the other hand, can manifest various display methods according to the magnitude of electric quantity. In the present invention, the electric quantity display module 14 uses a fan-shaped white light to show the intensity of electrotherapy energy, an orange light to show the status of electrotherapy, and a blue light to identify the magnitude of electric quantity. Moreover, an end of the electric connecting wire 13 is provided with a butting connector 130 (can be a USB 2.0 plug, a USB 3.0 plug, a MICRO USB plug or an AUDIO JACK plug) to connect electrically the connector 16, and the other end of the electric connecting wire 13 is provided at least with an electric current transmission element 132, with the electric current transmission element 132 being provided with a conductive patch 134.

The abovementioned electric current control module 15 includes at least a control element 150 and a circuit board 152 which is connected with the control element 150. The circuit board 152 is connected with the electric accumulator 12 and is installed with a processor 154, a power amplifier 156 connecting the processor 154, a transformer 158 connecting the power amplifier 156, and a g-sensor 159 installed on the circuit board 152 and connecting electrically the processor 154. The circuit board 152 is also provided with a first wireless transmitter 157 (can be a Bluetooth device) which is connected electrically with the processor 154. In addition, a second wireless transmitter 20 (can be a Bluetooth device) built on the terminal management equipment 2 is butted with the first wireless transmitter 157, and the terminal management equipment 2 is loaded with an application which can be an APP. Furthermore, the processor 154 is loaded with an AirSig application which is butted with the g-sensor 159, and the electrotherapy mode can be updated through a Bluetooth device wirelessly.

In the present embodiment, the electrotherapy machine 1 is built with the g-sensor 159. Therefore, the user can use the electrotherapy machine 1 to record the status of motion, with that the recording can be only carried out after the processor 154 has accomplished computing the messages generated by the g-sensor 159. In addition, the best combination of the record of motion, e.g. distance, speed, height and time of motion, with the usage information of electrotherapy, e.g. mode, location and time, can be known at any time to achieve a predetermined goal of health management.

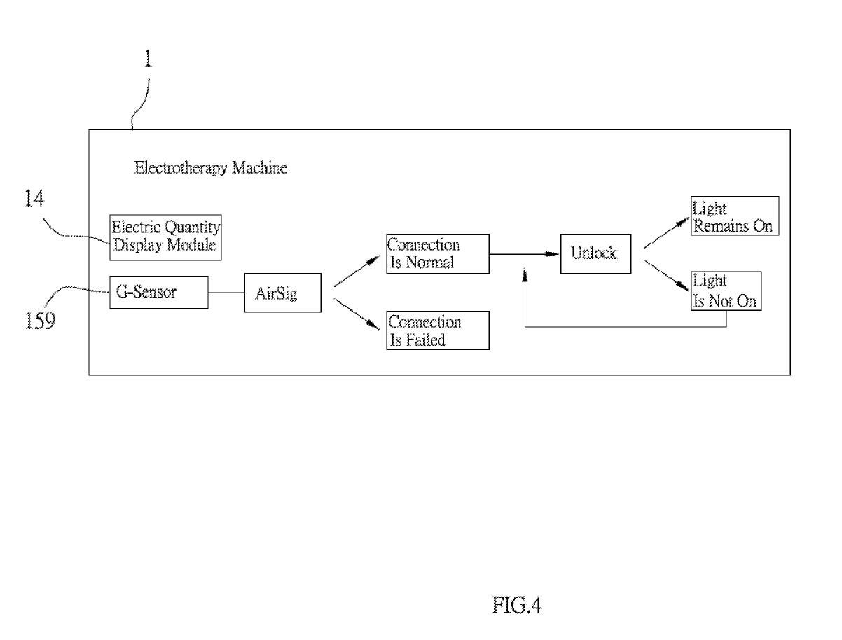

Furthermore, referring to FIG. 3 and FIG. 4, the purpose of recording the status of movement can be achieved by only using the abovementioned g-sensor 159. When the processor 154 is loaded with an AirSig application which is butted with the g-sensor 159, the electrotherapy machine 1 can be used as an authentication mechanism of Air-Signature, also achieving the effect of protecting privacy effectively and lowering significantly a concern that the personal data may be stolen. To use the AirSig application, a power-on button on the electrotherapy machine 1 is pressed down for a while to power on, and then an “E”-key and a “+”-key are pressed down simultaneously (referring to locations on FIG. 1) to activate the AirSig application. At this time, the electric quantity display module 14 on the electrotherapy machine 1 will tell whether the connection is successful or not by flashing or not flashing. When the connection is successful, the electric quantity display module 14 will flash and the user can use the g-sensor 159 in the electrotherapy machine 1 to induce the texts or graphs he or she sets up to wave in air. After unlocking, the electric quantity display module 14 will remain light-on. On the contrary, if the connection is failed, then the electric quantity display module 14 will light off. Accordingly, the AirSig application which is built into a smart phone or a tablet computer can be used to improve the security of personal data, preventing the personal data or related personal health records from being stolen.

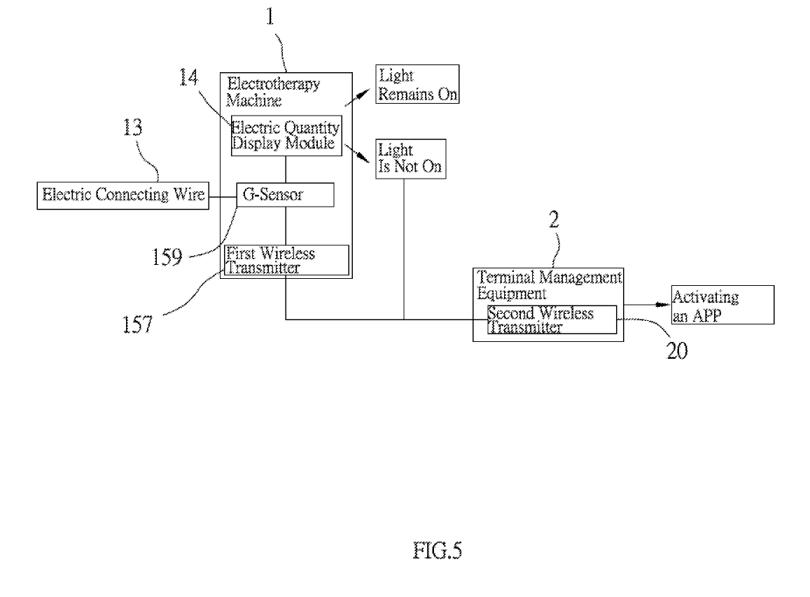

In addition, referring to FIG. 3 and FIG. 5, a second wireless transmitter 20 (can be a Bluetooth device) which is built on the terminal management equipment 2 is used primarily to butt with the first wireless transmitter 157. Hence, the personal health data, the health conditions after electrotherapy or the time and number of use through the electric connecting wire 13, which is accessed by the electrotherapy machine 1 through the g-sensor 159 when the user is in motion, can be transmitted from the first wireless transmitter 157 through the second wireless transmitter 20 to the terminal management equipment 2, and is recorded and managed using an APP loaded into the terminal management equipment 2, thereby achieving the best mode of health management. When using the terminal management equipment 2, the power-on button on the electrotherapy machine 1 is pressed down for a while to power on, and then the “E”-key and the “+”-key are pressed down simultaneously (referring to locations on FIG. 1) to activate the first wireless transmitter 157. In the same time, the second wireless transmitter 20 on the terminal management equipment 2 will be connected to the first wireless transmitter 157, and the electric quantity display module 14 will remain light-on when the connection is successful. On the contrary, the electric quantity display module 14 will light off if the connection is failed. After connection, data can be recorded and managed through the APP, roughly including personal information, activities to be done today, an activity log, experiences to be shared and software upgrade. Accordingly, by the APP which is built into a smart phone or a tablet computer, information can be transmitted directly and wirelessly with the electrotherapy machine 1 through a Bluetooth device, related data after electrotherapy can be recorded and the goal of health management can be achieved.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A smart portable electrotherapy machine, comprising:
a body, having a holding space and an installing opening connecting the holding space, wherein a surface of the body is provided with a display area and a control area;
an electric accumulator, which is installed in the holding space and connected electrically with a connector mounted at the installing opening;
an electric connecting wire, an end of which is provided with a butting connector connecting electrically the connector, and the other end of which is provided with an electric current transmission element;
an electric quantity display module, which is installed at the body, disposed in the display area and connected electrically with the electric accumulator;
an electric current control module, which is installed at the body, disposed in the control area and connected with the electric accumulator, with the electric current control module including a control element, a circuit board connecting the control element, a processor installed on the circuit board, a power amplifier connecting the processor, a transformer connecting the power amplifier, the circuit board being provided with a first wireless transmitter connecting electrically the processor, and an adequate electrotherapy mode being selected through the control of electric current;
a g-sensor, which is installed on the circuit board and connected electrically with the processor; and
a terminal management equipment, which is built with a second wireless transmitter butting with the first wireless transmitter and is loaded with an application, with the electrotherapy mode being updated wirelessly.

2. The smart portable electrotherapy machine, according to claim 1, wherein the application is a mobile application.

3. The smart portable electrotherapy machine, according to claim 1, wherein the processor is loaded with an AirSig application which is butted with the g-sensor, and the first wireless transmitter is a Bluetooth device.
